(19) United States
(12) Patent Application Publication
(10) Pub. No.: US 2025/0197817 A1
(43) Pub. Date: Jun. 19, 2025

(54) VIRUS PURIFICATION METHOD

(71) Applicant: The University of Tokyo, Tokyo (JP)

(72) Inventors: Takashi OKADA, Tokyo (JP); Yuji TSUNEKAWA, Tokyo (JP); Hiromi KINOH, Tokyo (JP); Mikako WADA, Tokyo (JP)

(73) Assignee: The University of Tokyo, Tokyo (JP)

(21) Appl. No.: 18/849,818

(22) PCT Filed: Mar. 23, 2023
(86) PCT No.: PCT/JP2023/011506

§ 371 Date: Sep. 23, 2024

(30) Foreign Application Priority Data
Mar. 24, 2022 (JP) 2022-047745

(51) Int. Cl. C12N 7/00

(52) U.S. Cl. CPC C12N 7/00; C12N 2750/14151

(57) ABSTRACT

It is an object of the present invention to provide a method for producing a virus, for use in obtaining viral particles with high purity, and the present method is more efficient than conventional methods. Specifically, the present invention relates to a method for obtaining a virus, comprising a step of treating a virus-containing sample with a surfactant. The surfactant may be one or more selected from the group consisting of an amphoteric surfactant, an anionic surfactant, a cationic surfactant, and a nonionic surfactant. In addition, the method for obtaining a virus according to the present invention may comprise a step of performing tangential flow filtration (TFF) to purify the virus.

### TECHNICAL FIELD

The present invention relates to a method for producing or purifying a virus.

### BACKGROUND ART

Gene therapy, in which genes or gene-introduced cells are administered into a human body for the purpose of treating diseases, is one of important therapeutic methods for the treatment of intractable diseases. At present, biological methods using viral vectors are mainly used as methods of introducing genes into mammalian cells for the purpose of gene therapy. A viral vector is a carrier for incorporating a gene to be introduced for the purpose of therapy into a viral strain that has lost or has partially lost the replicative ability and replicative ability of virus, so that the gene is efficiently introduced into cells and is allowed to express therein. Known viruses, from which viral vectors are derived, include enveloped viruses (viruses with envelopes) such as retrovirus, lentivirus, herpesvirus and Sendai virus, and non-enveloped viruses (viruses without envelopes) such as adenovirus and adeno-associated virus (AAV). Among them, AAV is used in gene therapy for the treatment of various diseases for such reasons that it can infect many types of cells, that it does not have pathogenicity to humans, and that the viral particles are physically stable.

By the way, in order to promote highly safe and highly effective gene therapy, it is necessary to prepare viruses used as vectors with high efficiency and high purification degree. In recent years, many adverse events have occurred with a massive systemic administration of AAV vectors, and thus, in order to ensure safety and efficacy, the importance of a method for producing and/or purifying an AAV vector with higher biological activity and less contamination of hollow particles has been increasing.

Methods for obtaining viral particles with high purity that have been known so far include: a method comprising culturing virus-producing cells under stress conditions in which the pH of a culture medium is increased, and increasing the ratio of viral particles released into the culture supernatant (Patent Literature 1); and a method comprising allowing virus-producing cells to come into contact with an acidic solution to obtain a highly purified virus (Non Patent Literature 2). These methods are reports regarding the improvement of a stage of obtaining viral particles from virus-producing cells.

On the other hand, as mentioned above, it is also extremely important to prepare viral particles with high purity and high concentration by reducing foreign matters as much as possible, and the purification process of viral particles also needs to be improved. Conventional methods of the concentration and purification of viral particles are complicated for mass preparation, and have a significant impact on biological activity, which poses significant challenges for industrial applications. For example, an affinity purification system using a VHH antibody with high affinity for AAV capsid has been in practical use for the purification of AAV, but low pH conditions during elution after adsorption have an influence upon the biological activity, and it is difficult to purify AAV in a state in which the biological activity is kept high. As such, at present, satisfactory methods for purifying viral particles have not yet been reported, and further improvement is needed.

### CITATION LIST

### Patent Literature

Patent Literature 1: WO2000/14205
Patent Literature 2: WO2015/06272

### SUMMARY OF INVENTION

### Technical Problem

In view of the above-described circumstances, it is an object of the present invention to provide a method for obtaining a virus, wherein the method is more efficient than conventional methods and is applied to obtain viral particles with high purity. More specifically, it is a method for producing or obtaining viral particles, comprising a step of efficiently purifying viral particles produced from virus-producing cells with high purity.

### Solution to Problem

The present inventors have cultured virus-producing cells, have then used CHAPS, a surfactant (amphoteric surfactant) that forms amphoteric ionic micelles, in combination with deoxycholic acid, an anionic surfactant, to treat a culture supernatant comprising AAV, and have then introduced a filtration operation by tangential flow filtration (TFF; alias: cross flow filtration), and thereby, the present inventors have found conditions for purifying a high-purity virus.

The AAV purified by the above-described method retains its original biological activity. In addition, it has been confirmed by electron microscopic observation that, unexpectedly, the hollow particles of AAV are preferentially destroyed upon implementation of the above-described method. Therefore, according to the above-described method, hollow particles, which have been hardly separated and removed by column chromatography used in the purification of AAV so far, can be efficiently reduced, and thus, it is possible to prepare AAV of extremely high quality.

Specifically, the present invention includes the following (1) to (8).
(1) A method for obtaining a virus, comprising a step of treating a virus-containing sample with a surfactant.
(2) The method according to the above (1), wherein the surfactant is one or more selected from the group consisting of an amphoteric surfactant, an anionic surfactant, a cationic surfactant, and a nonionic surfactant.
(3) The method according to the above (1) or (2), further comprising a step of performing tangential flow filtration (TFF) to purify the virus.
(4) The method according to any one of the above (1) to (3), wherein the amphoteric surfactant is one or more selected from the group consisting of CHAPS, CHAPSO, NDSB-211, and NDSB-201.
(5) The method according to any one of the above (1) to (4), wherein the anionic surfactant is sodium cholate and/or sodium deoxycholate.
(6) The method according to any one of the above (1) to (5), which is characterized in that the virus is a non-enveloped virus.
(7) The method according to the above (6), wherein the non-enveloped virus is an adeno-associated virus vector.
(8) A method for producing a virus, comprising:
   (a) a step of culturing virus-producing cells,
   (b) a step of preparing a virus-containing sample from the virus-producing cells or the cell culture solution, and
   (c) a step of obtaining a virus from the virus-containing sample prepared in the step (b) by the method according to any one of the above (1) to (5).

It is to be noted that the preposition “to” used in the present description indicates a numerical value range including the numerical values located left and right of the preposition.

### Advantageous Effects of Invention

According to the present invention, a method for purifying and obtaining a high-purity virus with high biological activity is provided. Thereby, it becomes possible to improve the safety and efficacy of gene therapy using viral vectors.

### BRIEF DESCRIPTION OF DRAWINGS

FIG. 1 shows the results obtained by studying filtration conditions according to a TFF method. There are shown the results obtained by subjecting Sample a (Lane 1), Sample b (Lane 2), Sample c (Lane 3), Sample d (Lane 4), Sample e (Lane 5), Sample f (Lane 6), Sample g (Lane 7) and Sample h (Lane 8) shown in the Examples to SDS-PAGE. “M” indicates the electrophoretic results of the markers.

FIG. 2 shows the results obtained by studying the effects of a surfactant in a virus purification step. There are shown the results obtained by subjecting Sample i (Lane 1), Sample j (Lane 2), Sample k (Lane 3), Sample 1 (Lane 4) and Sample m (Lane 5) shown in the Examples to SDS-PAGE. “SM” indicates a sample before TFF filtration, and “M” indicates the electrophoretic results of the markers.

FIG. 3 shows the results obtained by treating AAV9 with a surfactant and then purifying the treated AAV9 by filtration according to a TFF method. There are shown the results obtained by subjecting Sample n (Lane 1) and Sample o (Lane 2) shown in the Examples to SDS-PAGE. “SM” indicates a sample before TFF filtration, and “M” indicates the electrophoretic results of the markers.

FIG. 4 shows the results obtained by studying the infectivity of a purified virus. The percentage of HEK cells infected with the virus (AAV1) in each sample is shown. “Sup” indicates the infection percentage of the virus in a culture supernatant of ZsGreen-expressing AAV1, “TFF” indicates the infection percentage of the virus in a sample obtained by treating the culture supernatant with a surfactant and then filtering it by TFF, “CsCl” indicates the infection percentage of the virus in a sample obtained by adding a CsCl solution to the culture supernatant and then preserving it for 72 hours, and “affinity” indicates the infection percentage of the virus in a sample obtained by subjecting the culture supernatant to affinity chromatography and then eluting with glycine hydrochloride (pH 2.0).

FIG. 5 shows the results of observation by transmission electron microscopy of purified AAV1 (negative staining).

FIG. 6 shows the results of observation by transmission electron microscopy of purified AAV9 (negative staining).

### DESCRIPTION OF EMBODIMENTS

Hereafter, the embodiments for carrying out the present invention will be described.

A first embodiment relates to a method for obtaining a virus, comprising a step of treating a virus-containing sample with a surfactant, and more specifically relates to a method for obtaining a virus, comprising a step of treating a virus-containing sample with one or more surfactants selected from the group consisting of an amphoteric surfactant, an anionic surfactant, a cationic surfactant, and a nonionic surfactant. Specifically, the first embodiment relates to a method for obtaining a virus of interest by purifying the virus with high purity according to a method comprising a step of treating a virus-containing sample, for example, a suspension of virus-producing cells, a cell culture solution containing viruses, or a sample obtained by roughly purifying viruses from such a suspension or a cell culture solution (i.e. a sample also containing foreign matters other than viruses), with a surfactant.

The viruses according to the present embodiment include, but are not limited to, wild-type viruses, inactivated viruses (e.g., inactivated vaccine antigens, etc.), virus-like particles (VLPs) without genetic information, and viruses carrying foreign genes used as vectors (also referred to as “viral vectors”). In addition, the types of viruses are not particularly limited, and both enveloped viruses and non-enveloped viruses are included.

The enveloped virus is a virus whose viral genome and protein shell called capsid are covered by a membrane-like structure (envelope), whereas the non-enveloped virus is a virus without an envelope. Examples of the known envelope virus may include: DNA viruses such as herpesviruses, poxviruses, and hepadnaviruses; and RNA viruses such as flaviviruses, togaviruses, coronaviruses, orthomyxoviruses, paramyxoviruses, rhabdoviruses, Bunyaviruses, and retroviruses. On the other hand, examples of the known non-enveloped virus may include: DNA viruses such as adenovirus, adeno-associated virus (AAV), and papillomavirus; and RNA viruses such as picornavirus, calicivirus, norovirus, and rotavirus.

Surfactant is a generic term for substances that have hydrophilic groups and lipophilic groups/hydrophobic groups in molecules thereof, and such surfactant has the function of uniformly mixing polar substances and non-polar substances by forming micellar or lamellar structures. In general, the surfactant is classified into an amphoteric surfactant, an anionic surfactant, a cationic surfactant, and a nonionic surfactant.

The amphoteric surfactant is a surfactant that has both anionic and cationic moieties in a molecule thereof, and exists as a cation, an amphoteric ion or an anion, depending on the pH of the solution. The anionic surfactant is a surfactant that becomes an anion when it is dissociated in water, and is known to have a carboxylic acid, sulfonic acid or phosphoric acid structure as a hydrophilic group. The cationic surfactant is a surfactant that becomes a cation when it is dissociated in water, and is known to have tetraalkylammonium as a hydrophilic group. The nonionic surfactant is a surfactant having a nonionized hydrophilic moiety in a hydrophilic part thereof, and there are known, as such nonionic surfactants, low molecular weight surfactants such as alkyl glycoside and high molecular weight surfactants such as polyethylene glycol or polyvinyl alcohol.

The surfactant used in the present embodiment is not particularly limited, and can be selected, as appropriate, by those skilled in the art.

Examples of the amphoteric surfactant may include CHAPS (3-[(3-Cholamidopropyl)dimethylammonio]-1-propanesulfonate, CHAPSO (3-[(3-Cholamidopropyl)dimethylammonio]-2-hydroxy-1-propanesulfonate, NDSB-211 (3-[(2-Hydroxyethyl)dimethylammonio]propane-1-sulfonate, NDSB-201 (3-(1-Pyridinio) propanesulfonate, and NDSB-256-4T (3-(4-tert-Butyl-1-pyridinio) propanesulfonate.

Examples of the anionic surfactant may include sodium cholate and sodium deoxycholate.

Examples of the cationic surfactant may include benzalkonium chloride and benzethonium chloride.

Examples of the nonionic surfactant may include octylphenol ethoxylate (Triton X Series (product name)), polyoxyethylene sorbitan monolaurate (Tween (product name)), and octyl glucoside.

The treatment of a virus-containing sample with a surfactant can be carried out by adding the surfactant to the sample so that the final concentration of the surfactant becomes a desired concentration, and then reacting them. The final concentration of the surfactant is different depending on the surfactant used, but those skilled in the art can determine the optimal final concentration of the surfactant through preliminary experiments. For example, when CHAPS is used as an amphoteric surfactant and sodium deoxycholate is used as an anionic surfactant to obtain AAV, these surfactants may be added, such that the final concentration of each surfactant can be, for example, about 0.1% to 5.0%, about 0.2% to 1.5%, and preferably about 0.4% to 1.0%. The conditions under which the surfactants are added to the virus sample and they are reacted with one another may be conditions under which the effects of the surfactants added can be exhibited. Although the conditions are not particularly limited, the temperature conditions are, for example, 25° C. to 45° C., 30° C. to 40° C., and preferably 35° C. to 38° C., and the reaction time is, for example, several minutes to several hours, about 10 minutes to 1 hour, and preferably about 30 minutes.

The present embodiment may include a step of performing the removal of foreign matters and/or concentration of the virus by filtering a virus-containing sample according to a tangential flow filtration (TFF) method. The TFF method is a filtration method, in which a liquid containing a product of interest (in the present embodiment, a virus) is delivered horizontally along the surface of a filtration membrane for filtration. The TFF method differs from a normal flow filtration (NFF) method, in which a liquid is delivered perpendicular to the surface of a filtration membrane for filtration, in terms of the direction of delivering a liquid.

When filtration is carried out by the TFF method, it is necessary to select a filtration membrane with appropriate membrane pores. Since filtration membranes (hollow fiber membranes) with various materials and membrane pores are commercially available, an appropriate filtration membrane can be easily obtained by those skilled in the art. In general, the particle size of viruses is about 10 nm to 300 nm, and for example, the particle size of AAV is said to be about 18 to 25 nm. Taking into consideration the particle size of such viruses, it is desired to select an appropriate filtration membrane. When foreign matters smaller than viral particles are removed, the filtration may be carried out by the TFF method using a hollow fiber membrane with pores smaller than the diameter of viral particles, and a circulating fluid containing the viral particles may be recovered. On the other hand, when foreign matters larger than viral particles are removed, the TFF method may be performed using a hollow fiber membrane with pores larger than the diameter of viral particles, and a filtrate containing the viral particles may be recovered.

In the present embodiment, filtration of a sample by the TFF method may be carried out multiple times, as necessary. The delivery of a sample solution may be carried out either under pressure or without pressure. With regard to various conditions for carrying out filtration of a sample by the TFF method (e.g., conditions such as the pore size of a filtration membrane, a liquid-delivering rate, and a temperature), those skilled in the art can select appropriate conditions by conducting preliminary experiments.

Regarding the treatment of a virus-containing sample with a surfactant and filtration of the virus-containing sample by the TFF method, it is preferable to treat a sample with low purity (e.g., a culture medium of virus-producing cells, etc.) with a surfactant, and then to filter the sample by the TFF method. However, the surfactant treatment and the filtration by the TFF method may be carried out several times, respectively, until the virus is finally obtained, and the surfactant treatment and the filtration by the TFF method may be carried out in any order.

A second embodiment relates to a method for producing a virus, comprising:
(a) a step of culturing virus-producing cells,
(b) a step of preparing a virus-containing sample from the virus-producing cells or the cell culture solution, and
(c) a step of obtaining a virus from the virus-containing sample prepared in the step (b) by the method according to the first embodiment.

In the present embodiment, the “virus-producing cells” are cells having an ability to produce the elements necessary for forming viral particles, so as to produce viruses. The virus-producing cells may be either cells that are artificially produced to be able to generate viruses, or cells that have been infected by viruses in the natural environment and have been able to produce the viruses. The virus-producing cells in the present embodiment are preferably artificially produced virus-producing cells, and particularly preferably, the virus is a non-enveloped virus.

The method of artificially producing virus-producing cells is different depending on the type of a virus, and has already been described in detail in many reviews, etc. Thus, for the method of artificially producing virus-producing cells, such reviews can be referred to. Here, only an outline of the method for producing viral vector-producing cells will be described.

In the case of producing viral particles that function as vectors, a plasmid, in which the region encoding the nonstructural protein of the virus (a protein involved in viral replication, etc.) and the region encoding the structural protein of the virus (a protein such as a capsid) are deleted, and instead, a gene of interest is inserted, a plasmid encoding both the nonstructural and structural proteins of the virus, a plasmid encoding other necessary genes, depending on the type of viral vector, and the like are introduced into any given cells, so that virus-producing cells can be produced.

For example, in the case of an AAV vector, a plasmid containing a gene of interest, a plasmid containing a gene encoding a Rep protein (a protein necessary for virus replication) or a Cap protein (a protein constituting a capsid), and a plasmid encoding an Ela protein, an E1b protein, an E2 protein, an E4 protein, etc. derived from adenovirus, are introduced into HEK293 cells, HEK293T cells, etc., so that AAV vector-producing cells can be produced.

The conditions for culturing virus-producing cells have already been known, and those skilled in the art can select appropriate conditions according to the type of the virus. For example, the cells may be cultured in a medium such as DMEM containing necessary supplements (growth factors, amino acids, etc.) and serum, and IMDM, at about 30° C. to 38° C., in a CO₂ concentration of about 5% to 10%, for several days to 20 days, although the culture conditions are not particularly limited thereto.

The virus-containing sample may be an extract obtained by extracting the virus from virus-producing cells, or a roughly purified extract thereof. For example, in the case of viruses released into a culture medium, a culture medium after culturing virus-producing cells may be recovered and used as a sample, and in the case of viruses accumulated in cells, the recovered virus-producing cells may be crushed by a freeze-thawing method, an ultrasonic crushing method, etc., and debris and the like may be removed, so that the resultant may be used as a sample. Since many reagents, kits and the like for preparing a virus-containing samples from virus-producing cells are commercially available, these reagents and kits may be used to prepare a sample.

Furthermore, a virus of interest can be obtained from a virus-containing sample, using the method according to the first embodiment. The virus produced by the method according to the second embodiment can be used for various purposes as a highly pure and biologically active virus.

When the present description is translated into English and the English description includes singular terms with the articles “a,” “an,” and “the,” these terms include not only single items but also multiple items, unless otherwise clearly specified from the context that it is not the case.

Hereinafter, the present invention will be further described in the following examples. However, these examples are only illustrative examples of the embodiments of the present invention, and thus, are not intended to limit the scope of the present invention.

### EXAMPLES

### 1. Preparation of Culture Supernatant of ZsGreen-Expressing AAV1 or ZsGreen-Expressing AAV9

HEK293 cells were seeded at a density of 40,000/cm² on a HYPERFlask (Corning), and were then cultured in 10% FBS/DMEM. Three days after the culture, pHelper (TaKaRa), pAAV ZsGreen (TaKaRa), and pR2C1 (a plasmid containing the Rep gene of AAV1 and an AAV1 serum-type Cap gene; TaKaRa) or pR2C9 (a plasmid containing the Rep gene of AAV9 and an AAV9 serum-type Cap gene) were transfected into the cells, and the thus obtained cells were then cultured for 11 days. Thereafter, a culture supernatant containing AAV1 or AAV9 was recovered. The recovered culture supernatant was subjected to a centrifugation treatment at 10,000×g for 15 minutes, and the obtained supernatant was passed through a 0.45 μm bottle top filter (Thermo Fisher). The resultant was used as a starting sample (a sample containing AAV1 or AAV9) in the subsequent experiment.

### 2. Studies Regarding Experimental Conditions for Filtration by TFF Method

Filtration by the TFF method was carried out employing KrosFlo (registered trademark) KR2i TFF System (Repligen), using a UF membrane (Sterile ReadyToProcess Hollow Fiber Cartridge, 500 kD, 0.5 mm i.d. fiber, size 4 M housing, AdvantaPure tubing, Cytiva), unless otherwise described.

### Condition 1

A sample (200 mL) (Sample a) after passing through a bottle top filter was filtered by TFF (at the primary hollow fiber outlet, no pressure was applied), so that it was concentrated 4-fold (liquid amount after TFF; 50 mL) (Sample b). Subsequently, the buffer of the sample after the concentration was exchanged with PBS by TFF (PBS in an amount 18 times the liquid amount after the concentration was used).

Immediately after the buffer exchange, no precipitates were observed, but precipitates were gradually observed after the buffer was left at rest at room temperature.

Next, the sample after the buffer exchange was subjected to ultrafiltration with Amicon Ultra-15 (fraction size; 100 K, Merck), so that it was concentrated 60-fold (Sample d). As a result, the filter became clogged.

### Condition 2

A sample (200 mL) (Sample b) after passing through a bottle top filter was filtered by TFF (at the primary hollow fiber outlet, a pressure (14.5 psi) was applied), so that it was concentrated 4-fold (liquid amount after TFF; 50 mL) (Sample c). Subsequently, the buffer of the sample after the concentration was exchanged with PBS by TFF (PBS in an amount 18 times the liquid amount after the concentration was used).

Immediately after the buffer exchange, precipitates were observed.

Next, the sample after the buffer exchange was subjected to ultrafiltration with Amicon Ultra-15 (fraction size; 100 K, Merck), so that it was concentrated 60-fold (Sample e). As a result, the filter became clogged.

### Condition 3

A sample (4.8 L) after passing through a bottle top filter was filtered by TFF (at the primary hollow fiber outlet, no pressure was applied), so that it was concentrated (liquid amount after TFF; 125 mL). Subsequently, the buffer of the sample after the concentration was exchanged with an HNM buffer (50 mM HEPES, 150 mM NaCl, and 1 mM MgCl₂; pH 7.4) by TFF (substituted with the HNM buffer in an amount 200 times the liquid amount after the concentration).

Immediately after the buffer exchange, precipitates were observed.

### Condition 4

A sample (220 mL) after passing through a bottle top filter was filtered by TFF (at the primary hollow fiber outlet, no pressure was applied), so that it was concentrated 2-fold (liquid amount after TFF; 110 mL). Subsequently, the buffer of the sample after the concentration was exchanged with the HNM buffer by TFF (substituted with the HNM buffer in an amount 100 times the liquid amount after the concentration).

After the buffer exchange, precipitates were hardly observed.

Next, the sample after the buffer exchange was subjected to ultrafiltration with Amicon Ultra-15 (fraction size; 100 K, Merck), so that it was concentrated 120-fold. Thereafter, the buffer was exchanged with PBS. As a result, clogging hardly occurred.

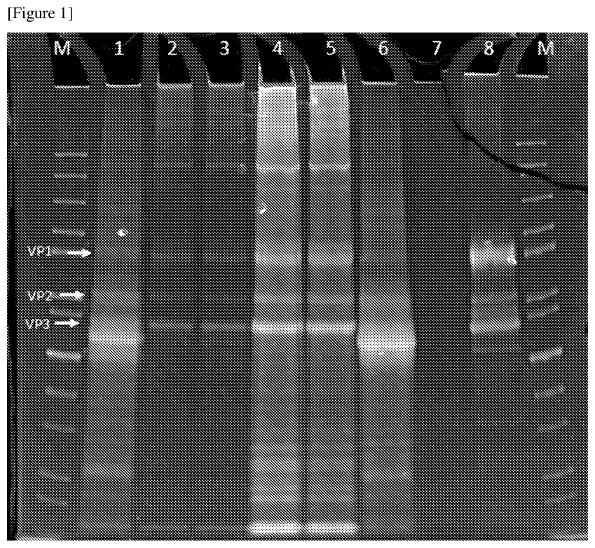

The above-described Samples a, b, c, d and e were subjected to SDS-PAGE, so that the remaining proteins were confirmed. Lanes 1, 2, 3, 4 and 5 in FIG. 1 correspond to Samples a, b, c, d and e, respectively. It could be confirmed that, by filtering a culture supernatant containing AAV1 by TFF, foreign matters other than viruses were removed, and VP1, VP2 and VP3, which are capsid proteins of AAV1, were relatively concentrated (Lanes 4 and 5 in FIG. 1).

Samples for SDS-PAGE were prepared by adding 10 μL of NuPAGE™ LDS Sample Buffer (4×) to 30 μL of Sample a, b, c, d and e, and heating the mixtures at 90° C. for 10 minutes. The heat-treated samples were placed on a electrophoretic gel (4% to 12% NuPAGE™, Bis-Tris, 1.0 mm, Mini Protein Gel, 10-well, Invitrogen), and specifically, a 35 μL/well sample and a 3 μL/well marker (PageRuler™ Unstained Protein Ladder, Thermo Fisher) were loaded on each well. After electrophoresis at CV of 200 V for 50 minutes, the gels were immersed in Oriole staining solution (Oriole Fluorescent Gel Stain, BioRad), and were then shaken for 90 minutes. Thereafter, the staining solution was replaced with water, and was then shaken for 10 minutes, and images were acquired with a ChemiDoc MP Imaging System (BioRad).

### 3. Studies Regarding Effects of Surfactants

### 3-1. Studies Using AAV1

In order to examine the effects of a surfactant on the purification efficiency (purity) of viral particles, the effects of an anionic surfactant, sodium deoxycholate, were first examined.

A culture supernatant containing AAV1 was centrifuged at 10,000×g for 15 minutes. The obtained supernatant was passed through a 0.45 μm bottle top filter. To the supernatant passed through the bottle top filter, sodium deoxycholate was added to a final concentration of 0.5%, and the obtained mixture was then stirred at 37° C. for 30 minutes (Sample f). The supernatant (220 mL) treated with sodium deoxycholate was filtered by TFF (at the primary hollow fiber outlet, no pressure was applied), so that it was concentrated 2-fold (liquid amount after TFF: 110 mL) (Sample g). Subsequently, the buffer of the sample after the concentration was exchanged with the HNM buffer by TFF (substituted with the HNM buffer in an amount 100 times the liquid amount after the concentration), and was then subjected to ultrafiltration with Amicon Ultra-15 (fraction size: 100 K, Merck), so that it was concentrated 120-fold (Sample h). As a result, clogging was hardly observed. The amount of foreign proteins in the obtained Sample h was confirmed, and as a result, it was found that the amount of foreign proteins was decreased by the sodium deoxycholate treatment.

Thus, the effects of other surfactants were also examined.

In addition to sodium deoxycholate that is an anionic surfactant, octyl glucoside as a nonionic surfactant and CHAPS as an amphoteric surfactant were examined in terms of their effects.

Untreated with Surfactants

A culture supernatant containing AAV1 was centrifuged at 10,000×g for 15 minutes. The obtained supernatant was passed through a 0.45 μm bottle top filter. The supernatant (205 mL) passed through the bottle top filter was filtered by TFF (at the primary hollow fiber outlet, no pressure was applied), so that it was concentrated (liquid amount after TFF: 50 mL). Subsequently, the buffer of the sample after the concentration was substituted with the HNM buffer by TFF (substituted with 2 L of the HNM buffer), and the primary side of the hollow fibers was washed three times (the liquid amount after the washing was 165 mL) (Sample i).

### Nonionic Surfactant

A culture supernatant containing AAV1 was centrifuged at 10,000×g for 15 minutes. The obtained supernatant was passed through a 0.45 μm bottle top filter. To the supernatant passed through the bottle top filter, octyl glucoside used as a nonionic surfactant was added to a final concentration of 0.5%, and the obtained mixture was then stirred at 37° C. for 30 minutes. The supernatant (212 mL) treated with octyl glucoside was filtered by TFF (at the primary hollow fiber outlet, no pressure was applied), so that it was concentrated (liquid amount after TFF: 50 mL). Subsequently, the buffer of the sample after the concentration was substituted with the HNM buffer by TFF (substituted with 2 L of the HNM buffer), and the primary side of the hollow fibers was washed three times (the liquid amount after the washing was 164 mL) (Sample j).

### Amphoteric Surfactant

A culture supernatant containing AAV1 was centrifuged at 10,000×g for 15 minutes. The obtained supernatant was passed through a 0.45 μm bottle top filter. To the supernatant passed through the bottle top filter, CHAPS used as an amphoteric surfactant was added to a final concentration of 0.5%, and the obtained mixture was then stirred at 37° C. for 30 minutes. The supernatant (219 mL) treated with CHAPS was filtered by TFF (at the primary hollow fiber outlet, no pressure was applied), so that it was concentrated (liquid amount after TFF: 46 mL). Subsequently, the buffer of the sample after the concentration was substituted with the HNM buffer by TFF (substituted with 2 L of the HNM buffer), and the primary side of the hollow fibers was washed three times (the liquid amount after the washing was 167 mL) (Sample k).

### Anionic Surfactant

A culture supernatant containing AAV1 was centrifuged at 10,000×g for 15 minutes. The obtained supernatant was passed through a 0.45 μm bottle top filter. To the supernatant passed through the bottle top filter, sodium deoxycholate used as an anionic surfactant was added to a final concentration of 0.5%, and the obtained mixture was then stirred at 37° C. for 30 minutes. The supernatant (214 mL) treated with sodium deoxycholate was filtered by TFF (at the primary hollow fiber outlet, no pressure was applied), so that it was concentrated (liquid amount after TFF: 45 mL). Subsequently, the buffer of the sample after the concentration was substituted with the HNM buffer by TFF (substituted with 2 L of the HNM buffer), and the primary side of the hollow fibers was washed three times (the liquid amount after the washing was 160 mL) (Sample 1).

### Amphoteric Surfactant+Anionic Surfactant

A culture supernatant containing AAV1 was centrifuged at 10,000×g for 15 minutes. The obtained supernatant was passed through a 0.45 μm bottle top filter. To the supernatant passed through the bottle top filter, CHAPS used as an amphoteric surfactant was added to a final concentration of 1% and sodium deoxycholate used as an anionic surfactant was added to a final concentration of 0.5%, respectively, and the obtained mixture was then stirred at 37° C. for 30 minutes. The supernatant (238 mL) treated with the surfactants was filtered by TFF (at the primary hollow fiber outlet, no pressure was applied), so that it was concentrated (liquid amount after TFF: 25 mL). Subsequently, the buffer of the sample after the concentration was substituted with the HNM buffer by TFF (substituted with 2 L of the HNM buffer), and the primary side of the hollow fibers was washed three times (the liquid amount after the washing was 172 mL) (Sample m).

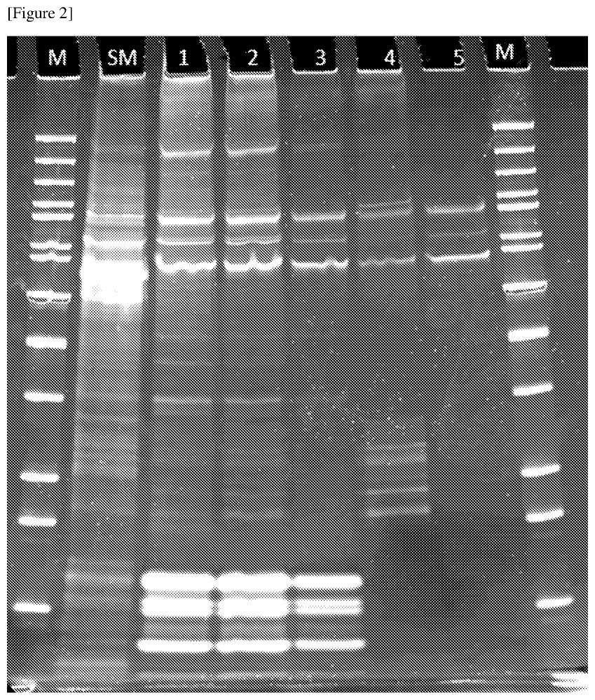

The above-described Samples i, j, k, l and m were subjected to SDS-PAGE, so that the remaining proteins were confirmed. Lanes 1, 2, 3, 4 and 5 in FIG. 2 correspond to Samples i, j, k, l and m, respectively. It was confirmed that when a culture supernatant containing AAV1 was treated with the amphoteric surfactant (CHAPS) and the anionic surfactant (sodium deoxycholate) before filtration by TFF, foreign proteins were very effectively removed and the VP1, VP2 and VP3 of capsid proteins were highly concentrated (FIG. 2, Lane 5).

It is to be noted that the treatment and electrophoresis of the samples for SDS-PAGE were carried out under the conditions described in the above 2.

Moreover, the amount of HCP (host cell protein, i.e. host cell-derived protein) in Samples i, j, k, l and m was quantified using the HEK 293 Host Cell Protein ELISA Kit (Cygnus), and the amount of DNA was quantified using the Quant-iT PicoGreen dsDNA Assay Kit (Thermo Fisher). The quantitative results are shown in Table 1 below.

TABLE 1

| Sample | HCP amount [μg/mL] | DNA amount [μg/mL] |
|---|---|---|
| SM | 33.9 | 4099 |
| Sample i | 1.9 | 1796 |
| Sample j | 0.3 | 1722 |
| Sample k | 0.6 | 1144 |
| Sample l | 1.1 | 3408 |
| Sample m | 0.19 | 420 |

(In Table 1, “SM” indicates a sample that is untreated with a surfactant and before TFF filtration.)

As shown in Table 1, it could be confirmed that, compared with the untreated sample (Sample i), if the sample was treated with the amphoteric surfactant and the anionic surfactant (Sample m), both HSP and DNA were dramatically reduced, and that the amount of HSP was reduced to about 1/10 and the amount of DNA was reduced to about ⅕.

### 3-2. Studies Using AAV9

Next, the effects of surfactants on purification of AAV9, instead of AAV1, were examined.

A culture supernatant containing AAV9 was centrifuged at 10,000×g for 15 minutes. The obtained supernatant was passed through a 0.45 μm bottle top filter. To the supernatant passed through the bottle top filter, CHAPS used as an amphoteric surfactant was added to a final concentration of 1% and sodium deoxycholate used as an anionic surfactant was added to a final concentration of 0.5%, respectively, and the obtained mixture was then stirred at 37° C. for 30 minutes. The supernatant treated with the surfactants was filtered by TFF, so that it was concentrated. Subsequently, the buffer of the sample after the concentration was substituted with the HNM buffer by TFF, and the primary side of the hollow fibers was washed three times.

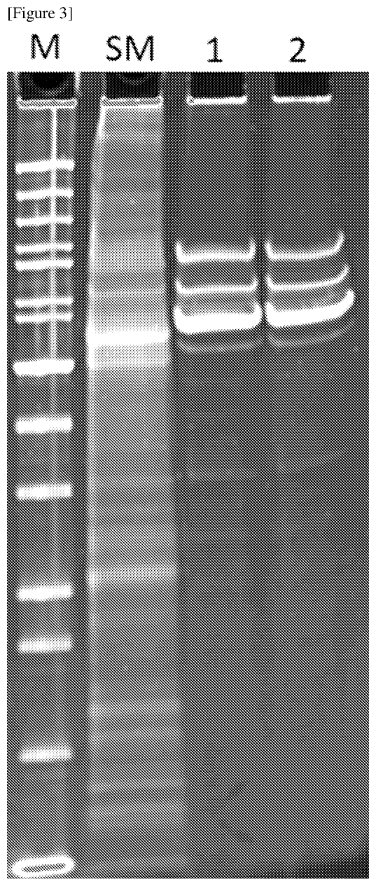

Herein, for the purpose of confirming whether or not the activity of the surfactant is lost when it is stored as a stock solution, the case of performing purification using a surfactant prepared at the time of use (Sample m) and the case of performing purification using a surfactant prepared from a stock solution (10% CHAPS and 10% sodium deoxycholate, each of which had been prepared 1 week before the use) (Sample o) were compared with each other, in terms of the degree of purification (FIG. 3). Lane 1 in FIG. 3 shows the results obtained using the surfactant prepared at the time of use, and Lane 2 shows the results obtained using the surfactant prepared from the stock solution.

From these results, it was found that the purification method using both an anionic surfactant and an amphoteric surfactant is effective not only for the purification of AAV1 but also for that of AAV9. In addition, it could be confirmed that the activity of the surfactants was not lost even if the surfactants were stocked as a solution, and that purification of viral particles with high purity was possible even when the surfactants prepared from such a stocked solution were used.

### 4. Studies Regarding Infectivity of Purified Virus

The infectivity of viruses purified by the method of the present invention was compared with that of viruses purified by conventional methods (a cesium chloride (CsCl) ultracentrifugation purification method and an affinity chromatography method).

The following samples were prepared: a culture supernatant (Sup) of ZsGreen-expressing AAV1: a solution (TFF) obtained by treating the culture supernatant with 1% CHAPS and 0.5% sodium deoxycholate at 37° C. for 30 min (stirring), then concentrating it by TFF, and then exchanging with an HNM buffer; as a sample imitating the CsCl ultracentrifugation purification method, a sample (CsCl) obtained by mixing a 1135.6 mg/mL CsCl solution and the culture supernatant at a ratio of 1:1, and then leaving the mixture at 4° C. for 2 days; and a sample (affinity) purified by affinity chromatography (POROS™ CaptureSelect™ AAVX Affinity Resin, Thermo Fisher) and then eluted with glycine hydrochloride (pH 2.0). The samples other than the TFF sample were substituted with the HNM buffer, using Amicon 100K, after the above-described treatment. These samples were added to HEK293 cells cultured in 12 wells to a density of 1×10⁹ vg/well (n=3), and were then cultured for 2 days. The cells in each well were peeled off, and the percentage of ZsGreen-positive cells was calculated by FACS.

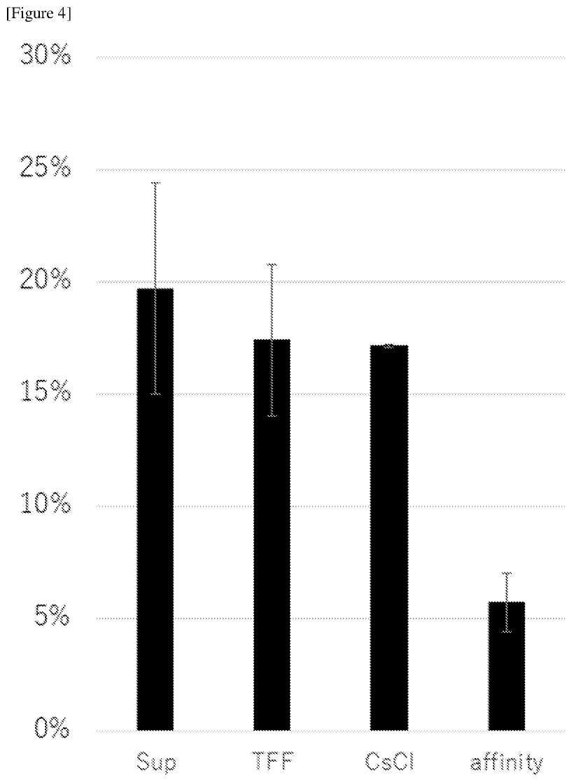

As shown in FIG. 4, it could be confirmed that the infectivity of the virus purified by the method of the present invention was almost the same level as that of the virus preserved under the same conditions as those for performing CsCl density gradient centrifugation (CsCl), and that the infectivity of the virus purified by the method of the present invention was much better than the virus purified by affinity chromatography (affinity).

### 5. Morphological Observation of Purified Viral Particles by Electron Microscopy

The morphology of AAV1 (Sample m) or AAV9 (Sample o), which had been treated with an amphoteric surfactant and an anionic surfactant and had been then purified by filtration through TFF, was observed by electron microscopy.

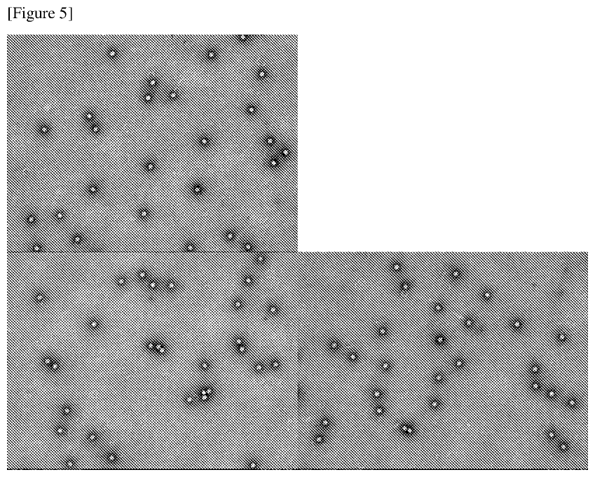

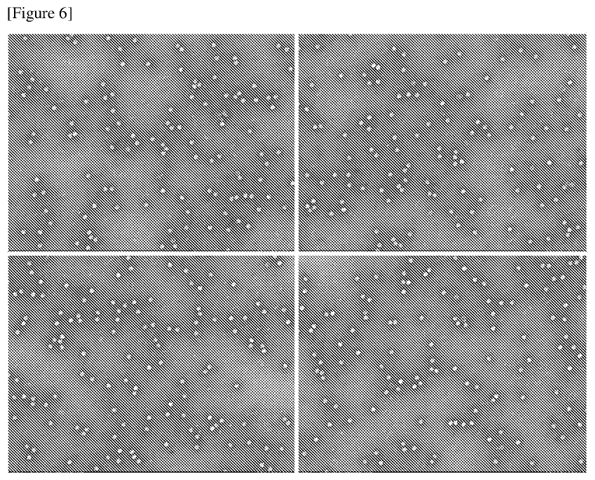

A collodion membrane (400 meshes, Cu) was hydrophilized (for 1.7 sec) by ion bombardment. Sample m or Sample o (3 μl) was placed on the hydrophilized collodion membrane and was then left at rest for 1 minute. A filter paper was pushed onto the edge of the collodion membrane to remove water. Thereafter, 3 μl of deionized distilled water (DDW) passed through a 0.22 μm filter was placed on the collodion membrane, and was then left at rest for 10 seconds. After that, excess water in the sample was removed by a filter paper. The step consisting of the DDW treatment and the removal of excess water with a filter paper were repeated twice in total. Thereafter, 3 μl of a staining solution was placed on the collodion membrane, and was then left at rest for 10 seconds. After that, a filter paper was pushed onto the edge the collodion membrane to remove excess water. The collodion membrane was left at rest for 2 minutes, and was then dried overnight in a desiccator, followed by microscopic observation. FIG. 5 shows microscopic images of the purified AAV1, and FIG. 6 shows microscopic images of the purified AAV9.

In the microscopic images of FIG. 5 and FIG. 6, hollow particles were hardly observed. Therefore, it is considered that the method of the present invention can efficiently reduce contamination of hollow particles, which is difficult to remove by the conventional column chromatography method.

### INDUSTRIAL APPLICABILITY

The present invention provides a method for efficiently preparing viruses (viral particles) such as viral vectors with high purity. Therefore, the present invention is expected to be utilized in the medical field and the like.

1. A method for obtaining a virus, comprising:
treating a virus-containing sample with a surfactant.

2. The method according to claim 1, wherein the surfactant is one or more selected from the group consisting of an amphoteric surfactant, an anionic surfactant, a cationic surfactant, and a nonionic surfactant.

3. The method according to claim 1, further comprising:
performing tangential flow filtration (TFF) to purify the virus.

4. The method according to claim 2,
wherein the surfactant is the amphoteric surfactant, and
wherein the amphoteric surfactant is one or more selected from the group consisting of CHAPS, CHAPSO, NDSB-211, and NDSB-201.

5. The method according to claim 2,
wherein the surfactant is the anionic surfactant, and
wherein the anionic surfactant is sodium cholate and/or sodium deoxycholate.

6. The method according to claim 1, wherein the virus is a non-enveloped virus.

7. The method according to claim 6, wherein the non-enveloped virus is an adeno-associated virus vector.

8. A method for producing a virus, comprising:
(a) culturing virus-producing cells,
(b) preparing a virus-containing sample from the virus-producing cells or a culture medium after culturing virus-producing cells, the cell culture solution, and
(c) obtaining a virus from the virus-containing sample prepared in the (b) by the method according to claim 1.

9. The method according to claim 3, wherein the surfactant comprises both an amphoteric surfactant and an anionic surfactant.

10. The method according to claim 9, wherein the amphoteric surfactant is one or more selected from the group consisting of CHAPS, CHAPSO, NDSB-211, and NDSB-201.

11. The method according to claim 9, wherein the anionic surfactant is sodium cholate and/or sodium deoxycholate.

12. The method according to claim 9, wherein the virus is a non-enveloped virus.

13. The method according to claim 12, wherein the non-enveloped virus is an adeno-associated virus vector.

14. A method for producing a virus, comprising:
(a) culturing virus-producing cells,
(b) preparing a virus-containing sample from the virus-producing cells or a culture medium after culturing virus-producing cells, and
(c) obtaining a virus from the virus-containing sample prepared in the step (b) by the method according to claim 9.
